(19) United States
(12) Patent Application Publication
(10) Pub. No.: US 2018/0325448 A1
(43) Pub. Date: Nov. 15, 2018

(54) ARTERY MAPPER

(71) Applicants: University of Washington, Seattle, WA (US); The Regents of the University of California, Oakland, CA (US)

(72) Inventors: Sheena M. Hembrador, Seattle, WA (US); Sepehr Rejai, Seattle, WA (US); Kevin Kadooka, Seattle, WA (US); Minoru Taya, Seattle, WA (US)

(73) Assignees: University of Washington, Seattle, WA (US); The Regents of the University of California, Oakland, CA (US)

(21) Appl. No.: 15/776,390

(22) PCT Filed: Nov. 16, 2016
(86) PCT No.: PCT/US2016/062238

§ 371 Date: May 15, 2018

(51) Int. Cl. A61B 5/00; A61B 5/026

(52) U.S. Cl. CPC A61B 5/489; A61B 5/0033; A61B 5/026; A61B 2562/046; A61B 5/7445; A61B 5/6843; A61B 2562/0247; A61B 5/681

(57) ABSTRACT

An artery mapper is attached over a target artery, and includes a sensor array that detects pressure changes corresponding to pulsatile flow through the artery. The signals from the sensor array are processed to identify signals having a frequency within a predetermined pulsatile range, and that define an elongate path across the sensor array. A display is provided directly over the sensor array. A digital controller circuit receives the signals from the detector array, and produces an image on the display directly over the detected signals, providing the practitioner with an image corresponding to the location of the artery, to facilitate cannulation of the artery.

### CROSS-REFERENCE TO RELATED APPLICATION

This application claims the benefit of Provisional Application No. 62/255,982, filed Nov. 16, 2015, the entire disclosure of which is hereby incorporated by reference.

### BACKGROUND

A challenge in patient care is arterial cannulation, that is, the insertion of a tube, e.g., a catheter or hypodermic needle, into a patient's artery. Arterial cannulation is a common procedure in various critical care settings. An arterial line, or A-line, for example, is a thin catheter inserted into an artery. Arterial lines are commonly used in intensive care medicine and anesthesia to monitor blood pressure and mean arterial pressure, and to obtain samples for arterial blood gas analysis.

An arterial line is usually inserted into the radial artery, but can alternatively be inserted into other arteries, for example, the brachial artery at the elbow, the femoral artery in the groin, the dorsalis pedis artery in the foot, or the ulnar artery in the wrist. Typically an over-the-wire or an over-the-needle technique is used for placement of the catheter, wherein insertion of the catheter into the artery is guided by a wire or needle, respectively.

Insertion of the catheter can be painful to the patient. Successful cannulation may be made difficult by the condition of the patient, for example hypotension, dehydration, and factors such as weight and the depth of the artery may interfere with accurately locating the desired artery. Multiple failed attempts can cause the artery to spasm making it virtually impossible to cannulate the artery.

Therefore it would be beneficial to accurately determine the location of the artery through noninvasive means prior to cannulation, and to provide the practitioner a visual indication of the artery location to facilitate accurate placement of the cannula. In particular, it would be beneficial to display the artery location for a length sufficient to allow the practitioner to determine the orientation of the artery, so that the needle or stylus can be positioned to intersect the artery generally or approximately along its axis. It is generally desirable to intersect the artery at an angle between about 30 degrees and 45 degrees. When the artery is suitably located, the practitioner may then align the needle at an angle suitable for insertion into the blood vessel.

Prior art systems, for example the systems for locating a blood vessel for cannulation have been disclosed. For example, U.S. Pat. No. 6,074,364, to Pam, which is hereby incorporated by reference in its entirety, discloses a blood vessel cannulation device that includes a pair of spaced-apart sensing guides configured to support ultrasonic probes to locate a blood vessel, and includes a cannula guide therebetween. However, it is relatively bulky, and requires probes that may not always be available or convenient to access.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

An artery mapper includes a sensor array configured to be attached to a skin surface overlying a target artery. The sensor array defines an array of detectors that are configured to generate signals responsive to a pressure or a change in pressure. A display device is disposed over the sensor array. A controller circuit is configured to receive signals generated by the sensor array, to identify from the received signals periodic pressure pulses that have a frequency within a predetermined frequency range corresponding to a pulsatile frequency and that define an elongate path across at least a portion of the sensor array, and to display on the display device an image that overlies the elongate path across the sensor array, such that the display shows a projection of the two-dimensional position of the artery below the display.

In an embodiment the sensor array is a capacitive sensor array. For example, the sensor array may include an insulating dielectric elastomer panel having a first plurality of electrodes on a first side and a second plurality of electrodes on a second side.

In an embodiment the first plurality of electrodes are parallel elongate electrodes oriented in a first direction on the elastomer panel, and the second plurality of electrodes are parallel elongate electrodes oriented in a second direction transverse to the first direction such that the first and second plurality of electrodes with the elastomer panel define an array of capacitors that generate the signals generated by the sensor array.

In an embodiment the first plurality of electrodes are electrically connected to a first multiplexer, and the second plurality of electrodes are electrically connected to a second multiplexer, and the multiplexers are controlled by the circuit to selectively scan the array of capacitors.

In an embodiment the sensor array is an array of piezoelectric detectors or an array of strain gauge detectors, and the display device is an LCD, LED, electrochromic, or electroluminescent display.

In an embodiment the circuit is a flex circuit and is disposed between the sensor array and the display device. In another embodiment the circuit is separate from, and releasably connectable to, the sensor array and/or the display device.

In an embodiment the sensor array is a capacitive sensor array having a dielectric elastomer panel, a first plurality of electrodes fixed on one side of the elastomer panel and a second plurality of electrodes fixed on an opposite side of the elastomer panel. The digital controller circuit includes a capacitive to digital converter configured to receive capacitive signals generated by the sensor array, and a microcontroller that receives digital signals from the capacitive to digital converter and identifies periodic signals within the predetermined frequency range that define an elongate path across the sensor array. For example, the predetermined frequency range of the periodic pressure pulse is 0.5 hertz to 3.5 hertz.

In an embodiment the sensor array is adhesively fixed to the skin surface.

An artery mapper includes a sensor array comprising an array of detectors configured to generate a signal responsive to an arterial pulse underlying the array of detectors, a display device disposed over the sensor array; and a digital controller circuit in signal communication with the array of detectors and configured to receive signals generated by the sensor array, and to identify from the received signals an elongate path corresponding to a projected position of the arterial pulse, and to display on the display device an image that overlies the elongate path.

In an embodiment the sensor array is a capacitive sensor array comprising an insulating dielectric elastomer panel having a first plurality of electrodes on a first side of the elastomer panel and a second plurality of electrodes on a second side of the elastomer panel. For example, the first plurality of electrodes may be parallel elongate electrodes oriented in a first direction on the elastomer panel, and the second plurality of electrodes may be parallel elongate electrodes oriented in a second direction transverse to the first direction such that the first and second plurality of electrodes with the elastomer panel define an array of capacitors that generate the sensor array signals.

In an embodiment the first plurality of electrodes are electrically connected to a first multiplexer, and the second plurality of electrodes are electrically connected to a second multiplexer, and the first and second multiplexers are controlled by the digital controller circuit to selectively scan the array of capacitors.

In an embodiment the sensor array comprises an array of piezoelectric detectors or an array of strain gauge detectors.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B illustrate an environmental view of an artery mapper in accordance with the present invention;

FIG. 2 is a functional diagram illustrating the artery mapper shown in FIGS. 1A and 1B;

FIG. 3 is a functional diagram illustrating another embodiment of an artery mapper in accordance with the present invention, wherein the circuit component is separate from, and connectable to, the sensor and display components; and

FIG. 4 illustrates a particular embodiment of the artery mapper shown in FIGS. 1A and 1B, and using a dielectric elastomer sensing array.

### DETAILED DESCRIPTION

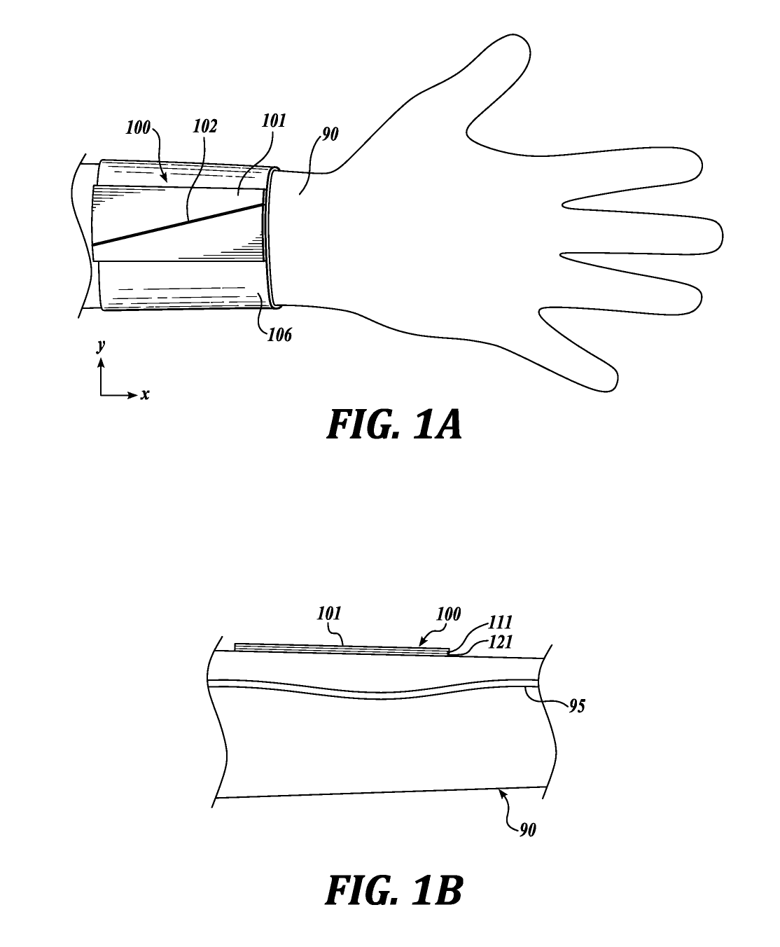

Referring to FIGS. 1A and 1B, an artery mapper 100 in accordance with the present invention, for assisting a practitioner in identifying the location and orientation of an artery 95 of a subject 90 will now be described. The artery mapper 100 will be a useful aid to medical practitioners during cannulation (the insertion of a tube or needle) of the artery 95. FIG. 1A is an environmental view of an artery detector and display, referred to herein as an artery mapper 100. The artery mapper 100 is first placed on the subject's wrist 90 and manually positioned to overlie the target artery, in this example the ulnar or radial artery 95. FIG. 1B shows diagrammatically a sectional side view of the artery mapper 100 on the wrist 90, and the underlying radial artery 95.

The artery mapper 100 includes a display 101 that is oriented to be visible to the practitioner during use. The display 101 may be, for example, a liquid crystal display, a light emitting diode display, an electrochromic display, an electroluminescent display, an e-paper display, or the like.

The display 101 produces an image 102 showing the two-dimensional location (i.e., projection) of the artery 95. The elongate image 102 on the display 101 is directly above the detected artery 95. In FIG. 1A the artery mapper 100 is attached to a strap 106 that extends around the wrist 90 to secure the artery mapper 100 at a desired location on the subject's wrist 90. For example, the strap 106 may be fastened with a conventional buckle, a hook-and-loop fastener such as Velcro®, or any conventional fastening mechanism, as is known in the art. A sensor array 121 is in contact with the subject's skin (or contacts the skin through a thin flexible membrane) over an area of the wrist 90 that is expected to overlie the target artery 95. In this embodiment a processing circuit assembly 111 is disposed between the sensor array 121 and the display 101. The two-dimensional image 102 is directly over the artery 95 to facilitate insertion of the cannula (not shown) into the artery 95.

Although in this embodiment the strap 106 secures the artery mapper 100 to the wrist 90, other attachment mechanisms are contemplated and may alternatively be used as are known in the art. Alternative attachment mechanisms may be preferable in particular applications. For example, an artery mapper 100 may be intended to locate and display the location and orientation of a target artery that is located in a location that is not amenable to a strap type attachment mechanism. In some embodiments the artery mapper 100 is adhesively attached to the subject. In other embodiments, the artery mapper 100 is attached to weighted members that are located on either side of the artery mapper 100 and sized to hang down on either side of the target anatomy, for example the wrist 90, to hold the artery mapper 100 in position. In another embodiment the artery mapper 100 includes a biased bracelet mechanism that engages the wrist 90 to secure the mapper 100 in a desired position. In another embodiment the artery mapper 100 includes tabs to facilitate manually holding the artery mapper 100 in place. Other attachment means are known in the art. In some embodiments a thin and flexible membrane (not shown) may be disposed between the artery mapper 100 and the subject's skin.

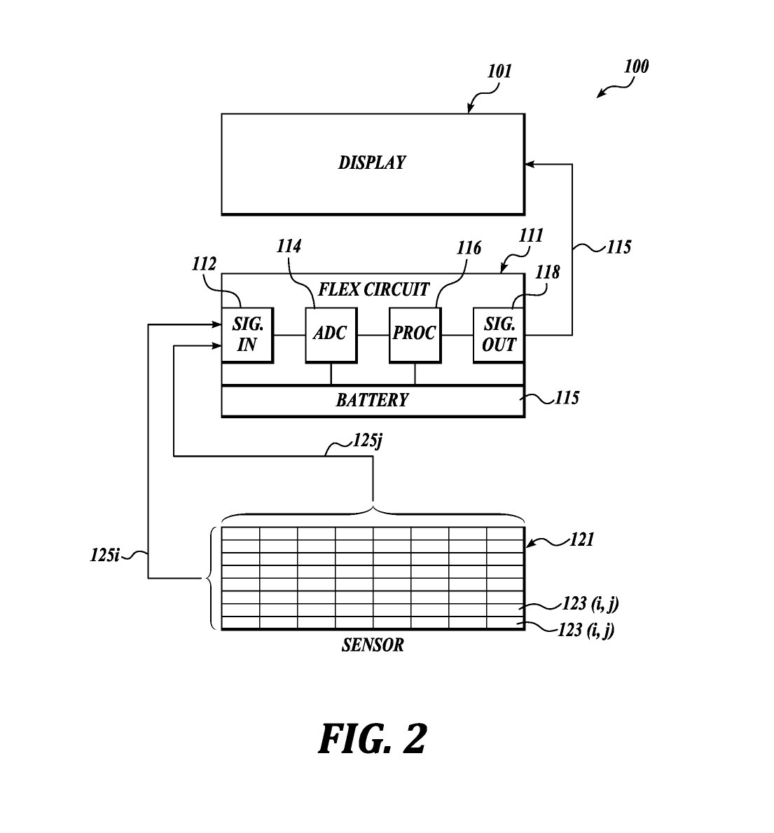

FIG. 2 illustrates diagrammatically an exploded functional diagram of the artery mapper 100. The artery mapper 100 includes the sensor array 121 that is configured to sense pressures or pressure changes at an array of locations 123(i,j). In this exemplary embodiment sixty-four sensing locations 123(i,j) are provided over a regular 8 by 8 rectangular grid. Other two-dimensional grid sizes and shapes are contemplated, and more or fewer sensing locations 123(i,j) may be used. For example, in another exemplary embodiment the sensor array defines 256 sensing locations on a 16 by 16 grid.

The sensor array 121 may comprise, for example, an array of piezoelectric sensors 123(i,j), as are well-known in the art. When pressure is applied across one axis of the piezoelectric crystal, thus compressing the lattice in one direction, that compression energy is converted into a voltage. The sensor array 121 in some embodiments comprises a thin layer piezoelectric crystal assembly that is sensitive enough to sense voltage differentials created by pulsations of the blood vessel 95 as detected from the surface of the skin of the subject 90.

In another embodiment the sensor array 121 is formed as an array of strain gauge sensors as are well-known in the art. In response to deflections or deformations produced by the pressure exerted from the pulsating flow in the artery 95, the electrical resistance of the strain gauge will change, producing a signal in the sensor array 121 that can be used to locate and image the artery 95. For example, a piezoresistive strain gauge uses the piezoresistive effect of bonded or formed strain gauges to detect strain due to applied pressure, resistance increasing as pressure deforms the material.

Referring also to FIG. 1A, the sensor array 121 is positioned to detect pressure, or changes in pressure, on the skin overlying the target artery 95, and to generate a signal corresponding to the detected pressure parameter. The signals 125i, 125j from the sensing locations 123(i,j) are transmitted to the circuit assembly 111. The circuit 111 may be a flex circuit 111 and may be located between the display 101 and the sensor array 121, as shown in FIG. 1B.

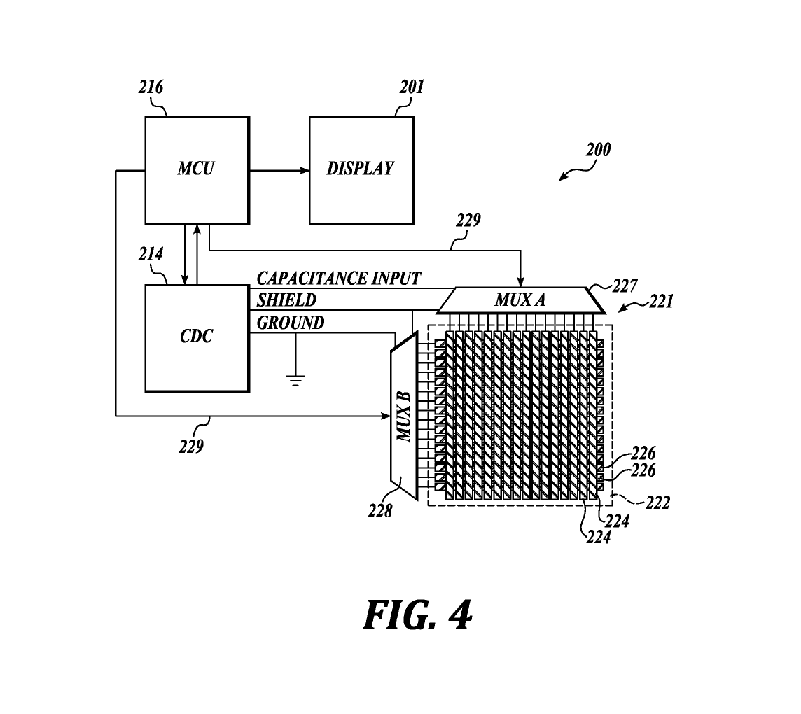

The circuit 111 in this embodiment includes a signal input 112 configured to receive the pressure signals 125i, 125j from the sensor array 121, a signal converter 114, for example a conventional analog to digital converter (ADC) 114 or a capacitance to digital converter (CDC) 214 (see FIG. 4). The signal converter 114 receives the signals from the input 112 and converts the signals to digital signals. The digital signals are transmitted to a microcontroller 116.

In a current embodiment the microcontroller 116 is configured to monitor for signals having a frequency that is within a range corresponding to an expected frequency associated with a pulse rate, for example between about 1 and 3 hertz, or between about 0.5 and 3.5 hertz. For example, the microcontroller 116 may be configured to convert the signals received from the signal converter 114 (for example, capacitance signals as discussed with reference to FIG. 4) from time to frequency domain by methods that are well-known in the art, for example fast Fourier transform (FFT), fast Hartley transform (FHT), or the like. When the artery mapper 100 is positioned over the target artery 95, signals within the predetermined frequency range that define an elongate region or path extending over at least a portion of the sensor array 121 indicates the location of the target artery 95 (in two dimensions).

The circuit 111 in this embodiment may include a power source, for example a battery 115 configured to power the display 101, other circuit components 111, and the sensor array 121. Alternatively, power may be provided from an external source. A signal output 118 outputs processed signals 115 that drive the display 101 to generate the desired image 102.

The display 101 is located directly over the sensor array 121, with the circuit 111 disposed between the display 101 and the sensor array 121. In this embodiment therefore, the artery mapper 100 includes three stacked layers, the sensor array 121, the flex circuit 111, and the display 101.

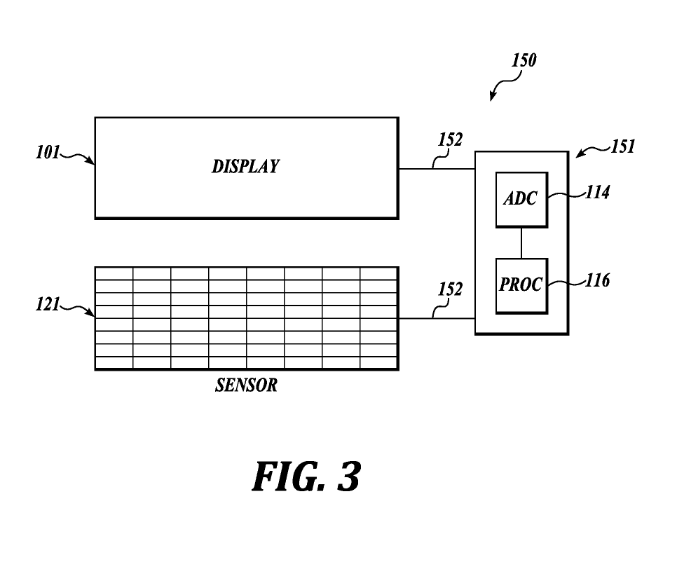

In another embodiment of an artery mapper 150 shown in FIG. 3, a circuit component 151 is disposed as a separate component that connects to the sensor array 121 and to the display 101 with cables 152 or wirelessly. This alternative construction may be preferable for example if the sensor 121 and the display 101 are intended to be a disposable product.

In the artery mappers 100, 150 the circuit assemblies 121, 151 are configured to use the data from the sensor array 121 to control the display 101 such that the displayed image 102 directly overlies the sensing locations 123(i,j) that detect the target frequency signals.

In another embodiment shown in FIG. 4 an artery mapper 200 includes an array of dielectric elastomer sensors 221. A conventional dielectric elastomer sensor is typically constructed by sandwiching a soft insulator material such as silicone between compliant electrodes, thereby producing a stretchable capacitor. The capacitance of the dielectric elastomer sensor is a function of the geometry of the electrodes, (e.g., the distance between the electrodes).

The artery mapper 200 shown in FIG. 4 includes a dielectric elastomer sensor array 221 that may conveniently define a plurality of capacitor elements on a unitary elastomer panel or member 222. The sensor array 221 includes an insulating dielectric elastomer member 222 (shown in phantom). A first plurality of spaced-apart, elongate electrodes 224, oriented vertically in FIG. 4 (eight shown) are formed or fixed on one side of the elastomer member 222. A second plurality of spaced-apart, elongate electrodes 226, oriented horizontally in FIG. 4, are formed or fixed on the opposite side of the elastomer member 222. The first plurality of electrodes 224 are connected to (or in signal communication with) a first multiplexer 227, and the second plurality of electrodes 226 are connected to (or in signal communication with) a second multiplexer 228.

A microprocessor, microcontroller, or the like 216 (hereinafter, microcontroller) is configured to provide control input 229 to the multiplexers 227, 228 to selectively monitor the respective electrodes 224, 226. For example, a four-input control 229 will allow the first and second multiplexers 227, 228 to selectively address sixteen electrodes 224 or 226, respectively. Therefore, the sixteen vertical electrodes 224 and sixteen horizontal electrodes 226 shown in FIG. 4 define a 16 by 16 array of capacitors, i.e., at the spaced-apart intersections of electrodes with the elastomer member 222 between the electrodes 224, 226 at each intersection.

The microcontroller 216 selectively scans the intersections by sequentially selecting the first electrodes 224 and the second electrodes 226 corresponding to each desired intersection. In some embodiments the microcontroller 216 may be operated to sequentially and methodically scan each intersection of electrodes 224, 226 from one corner of the sensor array 221 to a diagonally opposite corner. Alternative and more efficient scanning methods are contemplated. For example a transverse row of the sensor array 221 (e.g., a row that is intended to extend in the width direction of the wrist 90) may be scanned to locate one or more pulsatile signals, and subsequent transverse rows may be selectively scanned in sensor locations that are adjacent or near to a pulsatile signal detected in the preceding row.

As discussed above, the capacitance is a function of the geometry between the opposed electrodes 224, 226. During use the sensor array 221 is positioned on the skin, directly over the target artery 95. The pulsatile flow through the artery 95 produces a pressure or pressure change that causes a detectable change in the capacitance at intersections directly over the artery 95.

The microcontroller 216 is configured to systematically or selectively scan the capacitor locations defined by the intersections of the electrodes 224, 226. The corresponding signal output from each selected intersection of electrodes 224, 226 is communicated to a capacitance to digital converter 214, which digitizes the received signals and provides the digitized signals to the microcontroller 216. The microcontroller 216 is configured to identify the intersections that produce pulsatile signals indicating an artery, and to map the identified signals to the display 201, such that the display 201 produces an image directly over the detected artery 95.

In some embodiment the microcontroller 216 may be configured to systematically scan the sensor array 221 continuously, and to continuously update the display 201 based on the received signals. In other embodiments the microcontroller 206 may systematically scan the sensor array 221 once and display a static image based on the initial scan. In other embodiments the microcontroller 206 may scan the sensor array 221 periodically, for example once a minute or once every two minutes, and update the image on the display 201 after each scan.

While illustrative embodiments have been illustrated and described, it will be appreciated that various changes can be made therein without departing from the spirit and scope of the invention.

1. An artery mapper comprising:
a sensor array configured to be attached to a skin surface overlying a target artery, wherein the sensor array comprises an array of pressure detectors, wherein each pressure detector is configured to generate a signal responsive to a pressure or a change in pressure detected by the pressure detector;
a display device disposed over the sensor array; and
a digital controller circuit configured to:
(i) receive signals generated by the sensor array;
(ii) identify from the received signals periodic pressure pulses that have a frequency within a predetermined frequency range and that define an elongate path across at least a portion of the sensor array; and
(iii) display on the display device an image that overlies the elongate path across at least a portion of the sensor array.

2. The artery mapper of claim 1, wherein the sensor array is a capacitive sensor array.

3. The artery mapper of claim 2, wherein the capacitive sensor array comprises an insulating dielectric elastomer panel having a first plurality of electrodes on a first side of the elastomer panel and a second plurality of electrodes on a second side of the elastomer panel.

4. The artery mapper of claim 3, wherein the first plurality of electrodes comprise parallel elongate electrodes oriented in a first direction on the elastomer panel, and the second plurality of electrodes comprise parallel elongate electrodes oriented in a second direction transverse to the first direction such that the first and second plurality of electrodes with the elastomer panel define an array of capacitors that generate the signals generated by the sensor array.

5. The artery mapper of claim 4, wherein the first plurality of electrodes are electrically connected to a first multiplexer, and the second plurality of electrodes are electrically connected to a second multiplexer, and further wherein the first and second multiplexers are controlled by the digital controller circuit to selectively scan the array of capacitors.

6. The artery mapper of claim 1, wherein the sensor array comprises an array of piezoelectric detectors.

7. The artery mapper of claim 1, wherein the sensor array comprises an array of strain gauges.

8. The artery mapper of claim 1, wherein the display device comprises an LCD display or an LED display.

9. The artery mapper of claim 1, wherein the display device comprises an electrochromic display or an electroluminescent display.

10. The artery mapper of claim 1, wherein the digital controller circuit comprises a circuit disposed between the sensor array and the display device.

11. The artery mapper of claim 10, wherein the sensor array is a capacitive sensor array comprising a dielectric elastomer panel having a first plurality of electrodes fixed on one side of the elastomer panel and a second plurality of electrodes fixed on an opposite side of the elastomer panel, and the digital controller circuit comprises a capacitive to digital converter and a microcontroller, wherein the capacitive to digital converter is configured to receive capacitive signals generated by the sensor array and the microcontroller receives digital signals from the capacitive to digital converter and identifies periodic signals within the predetermined frequency range that define an elongate path across the sensor array.

12. The artery mapper of claim 1, wherein the artery mapper is configured to be adhesively attached to the skin surface.

13. The artery mapper of claim 1, wherein the predetermined frequency range of the periodic pressure pulse is 0.5 hertz to 3.5 hertz.

14. An artery mapper comprising:
a sensor array comprising an array of detectors configured to generate a signal responsive to an arterial pulse underlying the array of detectors;
a display device disposed over the sensor array; and
a digital controller circuit in signal communication with the array of detectors and configured to receive signals generated by the sensor array, and to identify from the received signals an elongate path corresponding to a projected position of the arterial pulse, and to display on the display device an image that overlies the elongate path.

15. The artery mapper of claim 14, wherein the sensor array is a capacitive sensor array.

16. The artery mapper of claim 15, wherein the capacitive sensor array comprises an insulating dielectric elastomer panel having a first plurality of electrodes on a first side of the elastomer panel and a second plurality of electrodes on a second side of the elastomer panel.

17. The artery mapper of claim 16, wherein the first plurality of electrodes comprise parallel elongate electrodes oriented in a first direction on the elastomer panel, and the second plurality of electrodes comprise parallel elongate electrodes oriented in a second direction transverse to the first direction such that the first and second plurality of electrodes with the elastomer panel define an array of capacitors that generate the sensor array signals.

18. The artery mapper of claim 17, wherein the first plurality of electrodes are electrically connected to a first multiplexer, and the second plurality of electrodes are electrically connected to a second multiplexer, and further wherein the first and second multiplexers are controlled by the digital controller circuit to selectively scan the array of capacitors.

19. The artery mapper of claim 14, wherein the sensor array comprises an array of piezoelectric detectors.

20. The artery mapper of claim 14, wherein the sensor array comprises an array of strain gauges.
